(19) United States
(12) Patent Application Publication
(10) Pub. No.: US 2006/0266365 A1
(43) Pub. Date: Nov. 30, 2006

(54) Vent cover for a mask assembly

(75) Inventor: Philip Thomas Stallard, Denistone East (AU)

Correspondence Address: NIXON & VANDERHYE, PC, 901 NORTH GLEBE ROAD, 11TH FLOOR, ARLINGTON, VA, 22203, US

(73) Assignee: ResMed Limited, Bella Vista (AU)

(21) Appl. No.: 11/443,098

(22) Filed: May 31, 2006

Related U.S. Application Data
(60) Provisional application No. 60/685,671, filed on May 31, 2005.

(51) Int. Cl. A62B 9/04; A61M 16/00; A62B 18/02

(52) U.S. Cl. 128/207.13; 128/202.27; 128/205.25; 128/206.28; 128/204.18; 128/205.11; 128/205.19; 128/207.11; 128/912

(57) ABSTRACT

A respiratory mask assembly for delivering breathable gas to a patient includes a gas delivery path defining at least one vent outlet and a connection flange surrounding the vent outlet. A vent cover is removably connected to the connection flange such that the vent cover is positioned in covering relation with respect to the vent outlet. The vent cover includes a cap portion providing at least one vent aperture for gas washout and a ring-shaped retaining portion defining an aperture into which the connection flange is inserted. The cap portion is movably connected to the retaining portion to allow the cap portion to be attached to and detached from the connection flange while the retaining portion remains connected to the connection flange.

### CROSS-REFERENCE TO RELATED APPLICATIONS

This application claims the benefit of U.S. Provisional Application No. 60/685,671, filed May 31, 2005, which is incorporated herein by reference in its entirety.

Also, U.S. patent application Ser. No. 10/390,826, filed Mar. 19, 2003, and U.S. patent application Ser. No. 10/655,621, filed Sept. 5, 2003, are each incorporated herein by reference in its entirety.

### FIELD OF THE INVENTION

The invention relates to a vent cover for use with a mask assembly for Non-invasive Positive Pressure Ventilation (NPPV) and for continuous positive airway pressure (CPAP) therapy of sleep disordered breathing (SDB) conditions such as obstructive sleep apnea (OSA).

### BACKGROUND OF THE INVENTION

Treatment of sleep disordered breathing (SDB), such as obstructive sleep apnea (OSA), by continuous positive airway pressure (CPAP) flow generator systems involves the continuous delivery of air (or other breathable gas) pressurized above atmospheric pressure to the airways of a human or other mammalian patient via a conduit and a mask. Typically, the mask fits over the mouth and/or nose of the patient. Pressurized air flows to the mask and to the airways of the patient via the nose and/or mouth. As the patient exhales, carbon dioxide gas may collect in the mask. A washout vent in the mask or conduit discharges the exhaled gas from the mask atmosphere.

The washout vent is normally located in the mask or near the mask in the gas delivery conduit coupled to the mask. The washout of gas through the vent to the atmosphere removes exhaled gases to prevent carbon dioxide build-up, and hence “rebreathing”, which represent a health risk to the mask wearer. Adequate gas washout is achieved by selecting a vent size and configuration that allows a minimum safe washout flow at a low operating CPAP pressure, which typically can be as low as 4 cm H₂O for adults and 2 cm H₂O for children.

Noise is a significant issue in CPAP treatment for the patient and/or the patient's bed partner. Excessive noise can lead to patients being non-compliant with the CPAP therapy. One source of noise is the exhaust through the vent in the mask or conduit. The flow of gas through the vent creates noise as it exits to and interacts with the atmosphere. Noise can adversely affect patient and bed-partner comfort, depending on both the magnitude and quality of the noise. Further, bi-level gas delivery regimes tend to generate more noise than do constant level gas delivery regimes. This is thought to be due to the extra turbulence created by the gas accelerating and decelerating as it cycles between relatively low and relatively high pressures in the bi-level gas delivery systems.

Fixed gas vents are known that have relatively low noise levels, which levels may be as low as 30 dBA at a therapy (mask) pressure of 12 cm H₂O. Such vents include, for example, the ResMed MIRAGE™ mask (disclosed in U.S. Pat. No. 6,561,190), the ResMed ULTRA MIRAGE™ mask (disclosed in U.S. Pat. No. 6,691,707), the ResMed VISTA™ mask (disclosed in U.S. Published Patent application 2003/0196657), the ResMed ACTIVA™ mask that includes an elbow with a vent (disclosed in International Patent Application PCT/AU03/01162 published as WO 2004/022147) and the ResMed MERIDIAN™ disposable nasal mask that includes an elbow incorporating a vent (disclosed in International Patent Application PCT/AU2004/000563). The contents of all of these patents and patent applications are incorporated herein by reference in their entireties.

There is a long felt and continuing need for quiet gas vents for masks and conduits, that are relatively inexpensive, simple in their construction and easy to maintain. Reducing the noise of gas being exhausted from a mask or conduit can significantly improve the user friendliness of the CPAP treatment. Providing a simple and easy to use low-noise vent can reduce the cost of CPAP treatments and thereby assist in making the treatment more affordable to patients suffering from SDB.

### SUMMARY OF THE INVENTION

One aspect of the invention relates to a vent cover that snaps onto an elbow of a mask assembly.

Another aspect of the invention relates to a vent cover constructed of polypropylene.

Another aspect of the invention relates to a vent cover that is retro-fit to an existing elbow of a mask assembly.

Another aspect of the invention relates to a vent cover designed such that the geometrical features are in a single line of draw for manufacture.

Another aspect of the invention relates to a vent cover that provides a tapered vent.

Another aspect of the invention relates to a vent cover that is easily accessible for cleaning.

Another aspect of the invention relates to a vent cover that is easy to assemble and disassemble.

Another aspect of the invention relates to a respiratory mask assembly for delivering breathable gas to a patient. The respiratory mask assembly includes a gas delivery path defining at least one vent outlet and a connection flange surrounding the vent outlet. A vent cover is removably connected to the connection flange such that the vent cover is positioned in covering relation with respect to the vent outlet. The vent cover includes a cap portion providing at least one vent aperture for gas washout and a ring-shaped retaining portion defining an aperture into which the connection flange is inserted. The cap portion is movably connected to the retaining portion to allow the cap portion to be attached to and detached from the connection flange while the retaining portion remains connected to the connection flange.

Another aspect of the invention relates to a respiratory mask assembly for delivering breathable gas to a patient. The respiratory mask assembly includes a gas delivery path defining at least one vent outlet and a connection flange surrounding the vent outlet. A vent cover is removably connected to the connection flange such that the vent cover is positioned in covering relation with respect to the vent outlet. The vent cover includes a first cap portion movably connected to a second cap portion. At least one of the first and second cap portions provides at least one vent aperture for gas washout. One of the first and second cap portions may be attached to and detached from the connection flange while the other of the first and second cap portions remains connected to the connection flange.

Another aspect of the invention relates a vent cover for a respiratory mask assembly that delivers breathable gas to a patient. The vent cover includes a cap portion and a ring-shaped retaining portion. The cap portion covers a vent outlet provided along a gas delivery path of the mask assembly. The cap portion includes at least one vent aperture for gas washout. The ring-shaped retaining portion defines an aperture structured to receive a connection flange surrounding the vent outlet. The cap portion is movably connected to the retaining portion to allow the cap portion to be attached to and detached from the connection flange while the retaining portion remains connected to the connection flange.

Another aspect of the invention relates to a vent cover for a respiratory mask assembly that delivers breathable gas to a patient. The vent cover includes a first cap portion and a second cap portion to cover a vent outlet provided along a gas delivery path of the mask assembly. One of the first and second cap portions provides at least one vent aperture for gas washout. The first cap portion is movably mounted to the second cap portion to allow one of the first and second cap portions to be attached to and detached from the connection flange while the other of the first and second cap portions remains connected to the connection flange.

Yet another aspect of the invention relates to a respiratory mask assembly for delivering breathable gas to a patient. The respiratory mask assembly includes a gas delivery path defining at least one vent outlet and a connection flange surrounding the vent outlet. A vent cover is molded in one piece from polypropylene and has a first portion movably connected to a second portion by a living hinge. One of the first and second portions provides at least one vent aperture for gas washout.

Yet another aspect of the invention relates to a respiratory mask assembly for delivering breathable gas to a patient. The respiratory mask assembly includes a gas delivery path defining at least one vent outlet and a connection flange surrounding the vent outlet. A vent cover provides at least one vent aperture for gas washout and is detachably connectable to the connection flange with a snap-fit.

Still another aspect of the invention relates to a method for mounting a vent cover to a respiratory mask assembly that delivers breathable gas to a patient. The method includes providing first and second vent portions, positioning the first vent portion adjacent to a connection flange surrounding a vent outlet of the mask assembly, and selectively connecting the second vent portion to the connection flange by moving the second vent portion relative to the first vent portion.

Other aspects, features, and advantages of this invention will become apparent from the following detailed description when taken in conjunction with the accompanying drawings, which are a part of this disclosure and which illustrate, by way of example, principles of this invention.

### BRIEF DESCRIPTION OF THE DRAWINGS

The accompanying drawings facilitate an understanding of the various embodiments of this invention. In such drawings:

FIG. 1 is a side view of a respiratory mask assembly including a vent cover constructed according to an embodiment of the present invention;

FIG. 2 is a perspective view of the vent cover and elbow removed from the respiratory mask assembly shown in FIG. 1, the cap portion of the vent cover in an operative position;

FIG. 3 is a cross-sectional view of the vent cover and elbow shown in FIG. 2;

FIG. 4 is a side view of the vent cover and elbow removed from the respiratory mask assembly shown in FIG. 1, the cap portion of the vent cover in an inoperative position;

FIG. 5 is a perspective view of the vent cover shown in FIG. 1 removed from the elbow;

FIG. 6 is a side view of a respiratory mask assembly including a vent cover constructed according to another embodiment of the present invention;

FIG. 7 is an perspective view of the vent cover and elbow removed from the respiratory mask assembly shown in FIG. 6, the cap portion of the vent cover in an operative position;

FIG. 8 is a cross-sectional view of the vent cover and elbow shown in FIG. 7;

FIG. 9 is an perspective view of the vent cover and elbow removed from the respiratory mask assembly shown in FIG. 6, the cap portion of the vent cover in an inoperative position;

FIG. 10 is another perspective view of the vent cover and elbow removed from the respiratory mask assembly shown in FIG. 9, the cap portion of the vent cover in an inoperative position;

FIG. 11 is an perspective view of the vent cover shown in FIG. 6 removed from the elbow;

FIG. 12 is another perspective view of the vent cover shown in FIG. 11 removed from the elbow;

FIG. 13 is a top view of the vent cover shown in FIG. 11;

FIG. 14 is a right side view of the vent cover shown in FIG. 11;

FIG. 15 is a front view of the vent cover shown in FIG. 11; and

FIG. 16 a left side view of the vent cover shown in FIG. 11.

### DETAILED DESCRIPTION OF ILLUSTRATED EMBODIMENTS

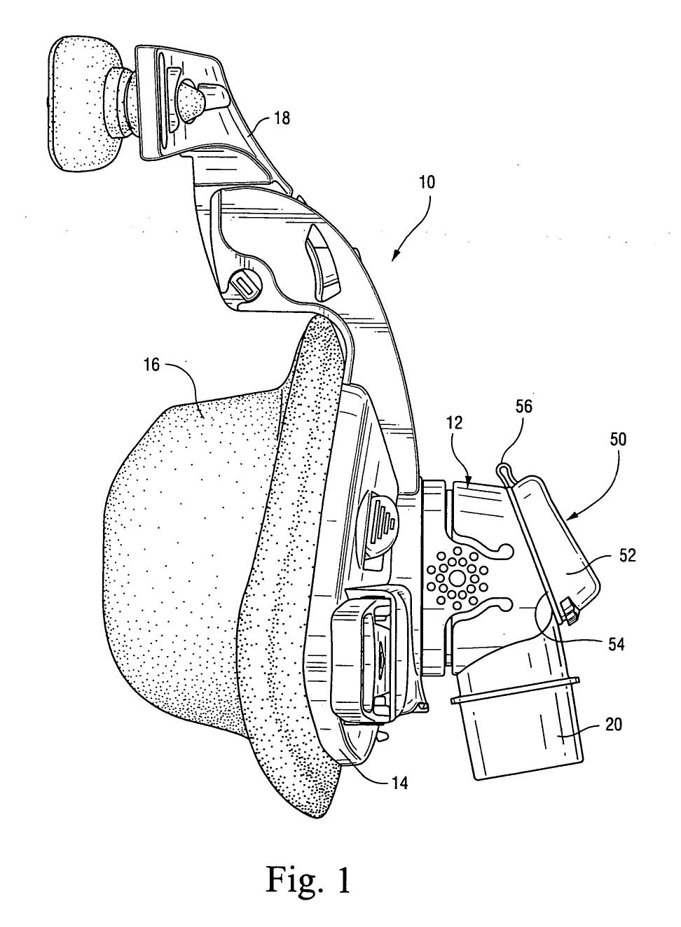

FIG. 1 illustrates a respiratory mask assembly 10 including a vent cover 50 constructed according to a first embodiment of the present invention. The vent cover 50 is adapted to be removably connected to an elbow 12 of the mask assembly 10 with a snap-fit. The vent cover 50 is constructed from polypropylene or another material suitable for snap-fits, and provides a low cost, single molding alternative to silicone vents that are known in the art. In use, the vent cover 50 is structured to direct exhaust air from the mask assembly 10 in manner that minimizes noise and avoids disturbance of a bed partner.

Mask Assembly

As shown in FIG. 1, the respiratory mask assembly 10 includes a frame 14 having a front surface and a rear surface adapted in use to face the patient. A cushion 16 may be permanently or removably connected to the rear surface of the frame 14. A forehead support 18 is mounted to an upper portion of the frame 14. A headgear assembly (not shown) may be removably attached to the frame 14 and/or forehead support 18 to maintain the frame 14 and cushion 16 in a desired adjusted position on the patient's face. A swivel elbow 12 is removably attached to the front surface of the frame 14. The elbow 12 is structured to be connected to an air delivery tube that delivers breathable gas to the patient.

In the illustrated embodiment, the mask assembly 10 is a nasal mask structured to deliver breathable gas to a patient's nose. Such a mask assembly is disclosed in U.S. patent application Ser. No. 10/655,621, filed Sept. 5, 2003, the entirety incorporated herein by reference. It is to be understood that the mask assembly 10 is merely exemplary, and the vent cover 50 may be adapted for use with other suitable mask assemblies. For example, the vent cover 50 may be designed for use with a full-face (oro-nasal) mask, a mouth (oro) mask, or a nasal mask.

Illustrated Embodiment of Swivel Elbow

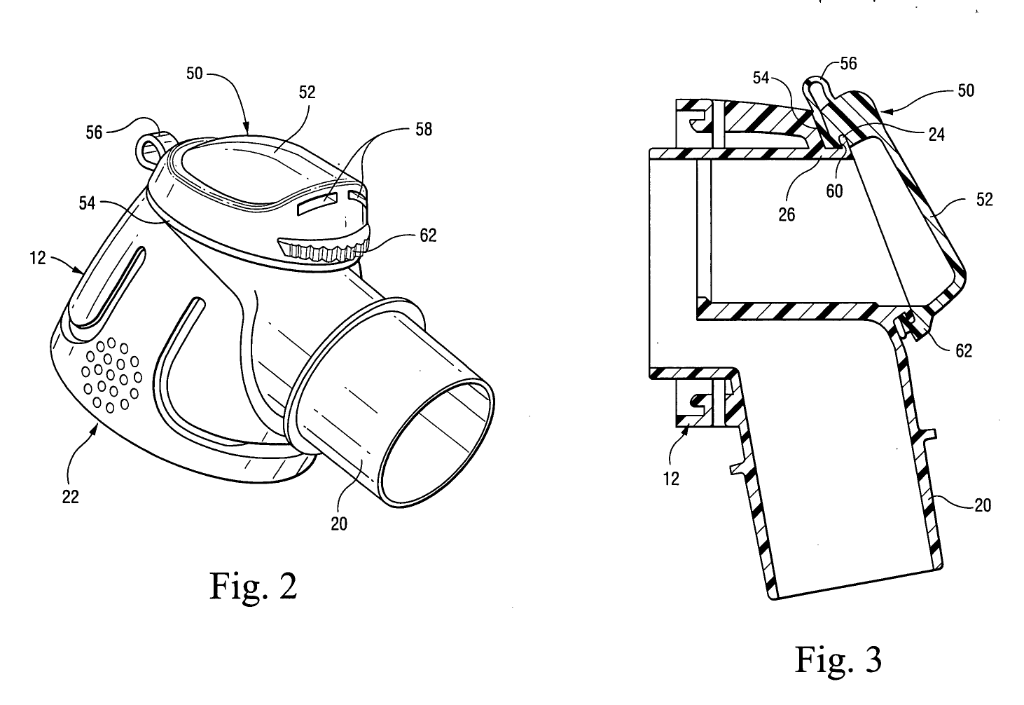

FIGS. 2 and 3 show an embodiment of the swivel elbow 12 in greater detail. The swivel elbow 12 is rotationally connected to the frame 14 and includes a stem 20 which is adapted to receive the air delivery tube to supply pressurized breathable air or gas from a blower (not shown). The end portion of the swivel elbow 12 includes a flexible quick release mechanism 22 to detachably connect the swivel elbow 12 to the frame 14. Also, the swivel elbow 12 includes a connection flange 24 provided at a distal end of a wall 26 surrounding the outlet of the exhaust port. The swivel elbow may include one or more vent openings at the outlet. An example of such a swivel elbow 12 is described in U.S. patent application Ser. No. 10/390,826, filed Mar. 19, 2003, and U.S. patent application Ser. No. 10/655,621, filed Sept. 5, 2003, the contents of which are incorporated in their entirety by reference herein. While aspects of the invention are described with reference to a swivel elbow, it is also applicable to fixed elbows or other swivel and/or fixed conduits.

First Illustrated Embodiment of Vent Cover

As shown in FIGS. 1-4, the vent cover 50 is adapted to be removably connected to the flange 24 provided on the elbow 12 of the mask assembly 10. The vent cover 50 is positioned in covering relation with respect to the vent opening at the outlet. The vent cover 50 may be retro-fit to an existing elbow of a mask assembly such as those described in U.S. patent application Ser. Nos. 10/390,826 and 10/655,621 noted above. Alternatively, the vent cover 50 may be fitted to a new elbow having such a connection flange. As described below, the vent cover 50 is structured such that it may be “snapped” onto the elbow 12 and easily accessed for cleaning.

As best shown in FIG. 5, the vent cover 50 includes a cap portion 52, a retaining portion 54, and an intermediate portion 56 that interconnects the cap portion 52 and the retaining portion 54. In the illustrated embodiment, the vent cover 50 is molded in one piece and the intermediate portion 56 acts as a living hinge to allow relative movement between the cap portion 52 and the retaining portion 54. The vent cover 50 may be molded from biocompatible polypropylene, or any other material suitable for snap-fits.

The front face of the cap portion 52 includes at least one vent aperture 58, e.g., two vent apertures, extending from an interior of the cap portion 52 to an exterior of the cap portion 52 for gas washout. The vent apertures 58 may have any suitable configuration as is known in the art. For example, the vent apertures 58 may be slots similar to those in the ResMed MERIDIAN™ disposable nasal mask (disclosed in International Patent Application PCT/AU2004/000563), tapered holes such as those in the ResMed ACTIVA™ mask (disclosed in U.S. patent application Ser. No. 10/655,621), and/or an arc shape such as that disclosed in U.S. Provisional Patent Application No. 60/643,114. The contents of these applications are incorporated in their entirety by reference herein.

The interior of the cap portion 52 includes a flange or aperture 60 (see FIG. 3) for engaging the connection flange 24 of the elbow 12. Also, the exterior of the cap portion 52 includes a grip 62, e.g., formed by a series of ridges, to facilitate assembly and/or disassembly of the cap portion 52 to the flange 24.

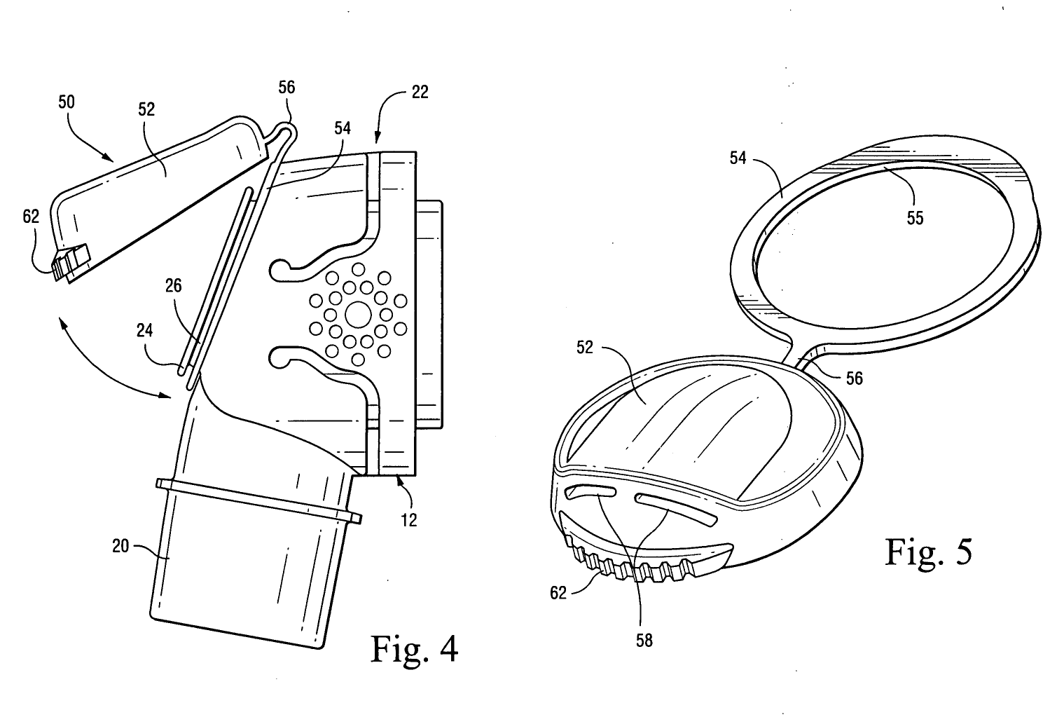

The retaining portion 54 is in the form of a ring that defines an aperture 55. The vent cover 50 is connected to the elbow 12 by placing the ring-shaped retaining portion 54 over the flange 24 such that the flange 24 is inserted through the aperture 55. This positions the retaining portion 54 around the wall 26 and below the flange 24 as shown in FIG. 4. The retaining portion 54 may resiliently deform to overcome the flange 24. The cap portion 52 is then moved relative to the retaining portion 54 toward the elbow 12 until the flange 24 of the elbow 12 removably engages the flange or aperture 60 of the cap portion 52 with a snap-fit (e.g., similar to a standard bottle cap). The cap portion may resiliently deform to snap onto and firmly grip the flange when the cap portion reaches its operative position as shown in FIGS. 1-3. The cap portion 52 may create an audible clip or snap when properly attached. A seal is maintained by the contact between the edge of the cap portion 52 and the lip of the elbow 12. When desired, the cap portion 52 may be moved into its inoperative position (see FIG. 4) wherein the cap portion 52 alone is detached from the elbow 12 to allow easy access to the interior vent profile, e.g., for cleaning.

Also, when attached, the vent cover 50 may be rotated with respect to the flange 24 to adjust the position or direction of the vent apertures 58.

Further, in an embodiment, the vent cover 50 may comprise the cap portion 52 only that may be detachably connected to the flange 24 with a snap-fit.

Second Illustrated Embodiment of Vent Cover

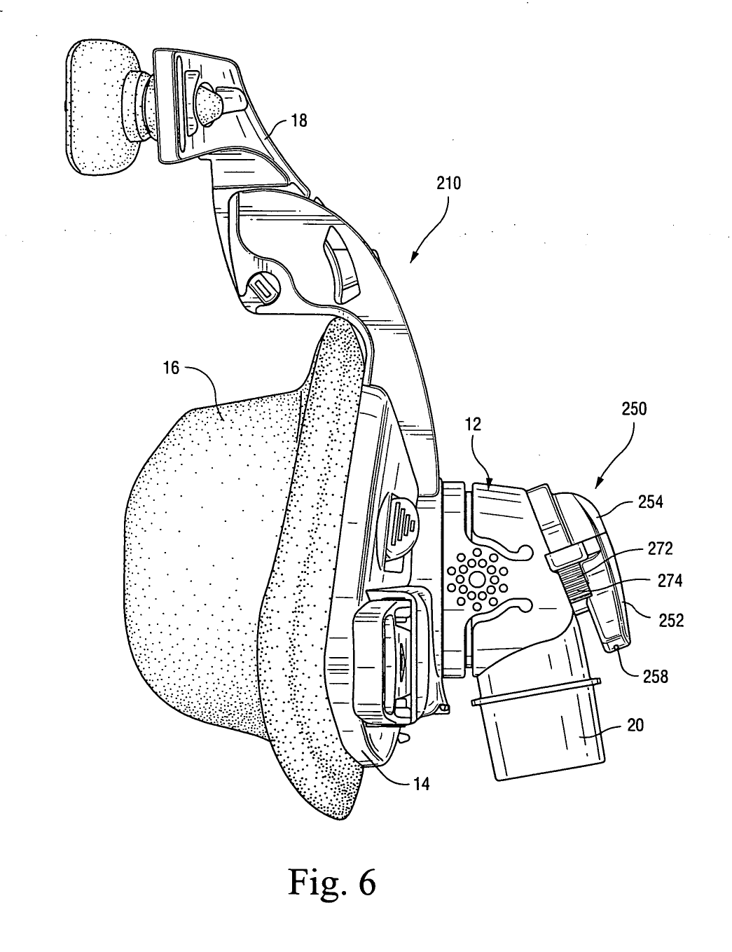

FIG. 6 illustrates a respiratory mask assembly 210 including a vent cover 250 constructed according to a second embodiment of the present invention. The vent cover 250 is adapted to be removably connected to an elbow 12 of the mask assembly 210. The mask assembly 210 and swivel elbow 12 thereof are substantially similar to those described above and indicated with similar reference numerals. As noted above, the mask assembly 210 is merely exemplary, and the vent cover 250 may be adapted for use with other suitable mask assemblies.

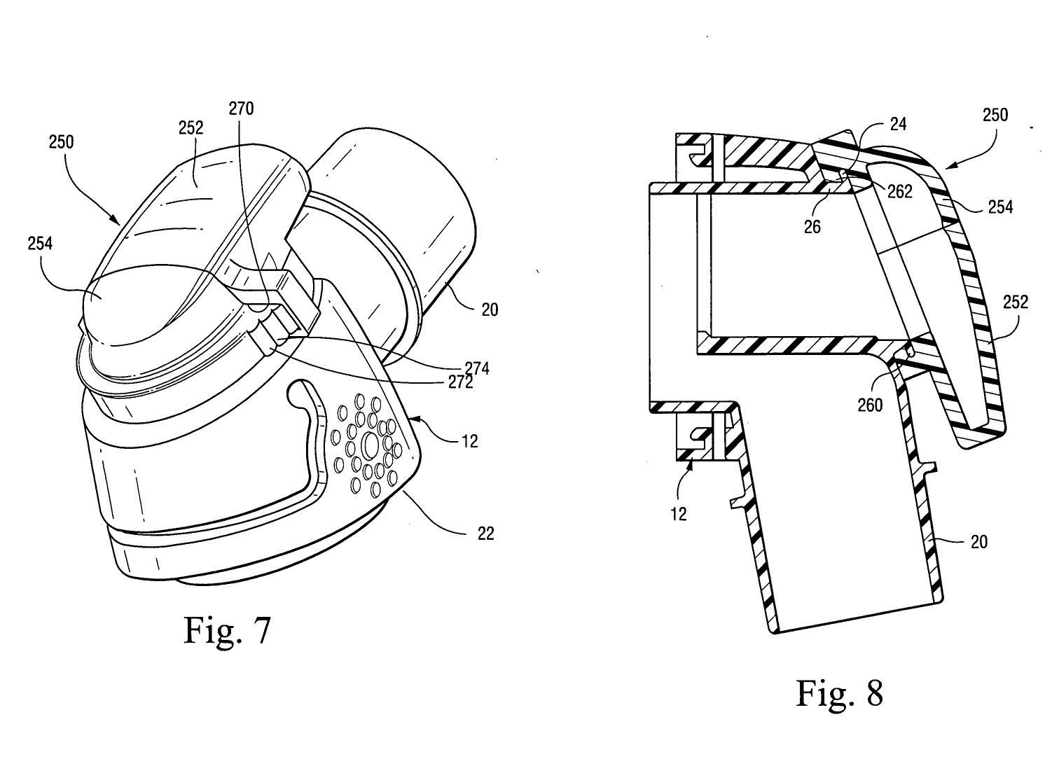

As shown in FIGS. 6-10, the vent cover 250 is adapted to be removably connected to the flange 24 provided on the elbow 12 of the mask assembly 210. The vent cover 250 is positioned in covering relation with respect to the vent opening at the elbow outlet. Similar to the above, the vent cover 250 may be retro-fit to an existing elbow or fitted to a new elbow having a connection flange. As described below, the vent cover 250 is structured such that it may be easily assembled to the elbow 12 and easily accessed for cleaning.

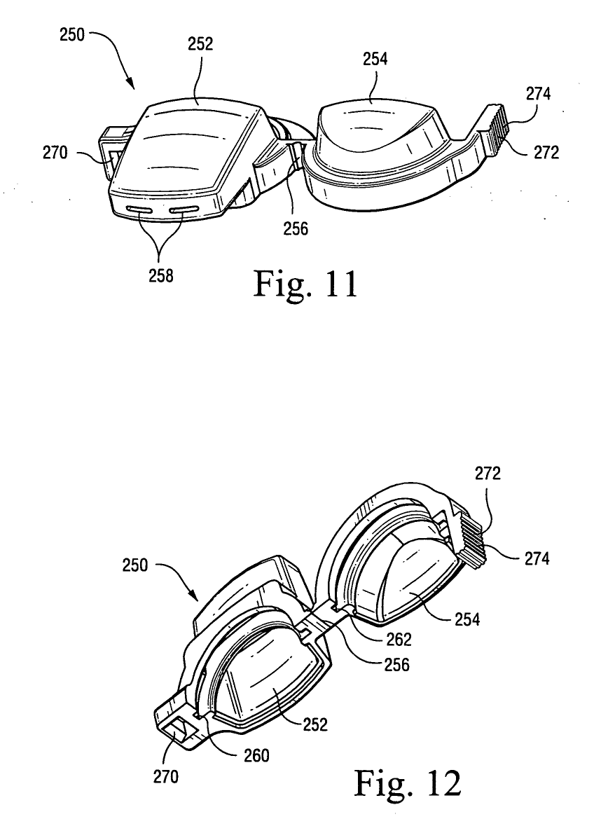

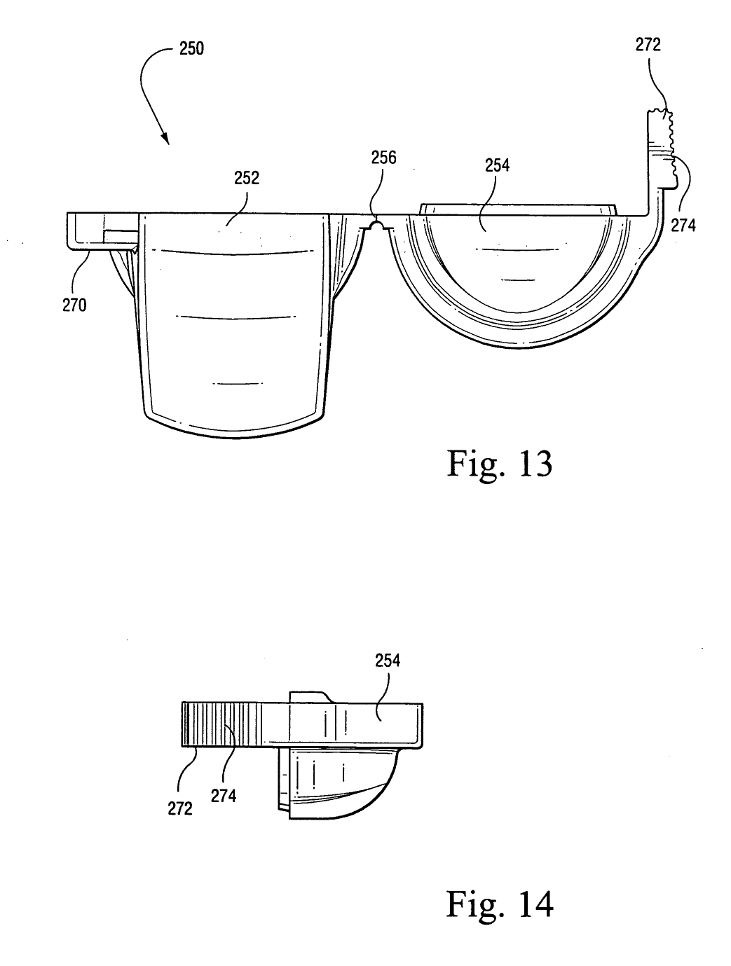

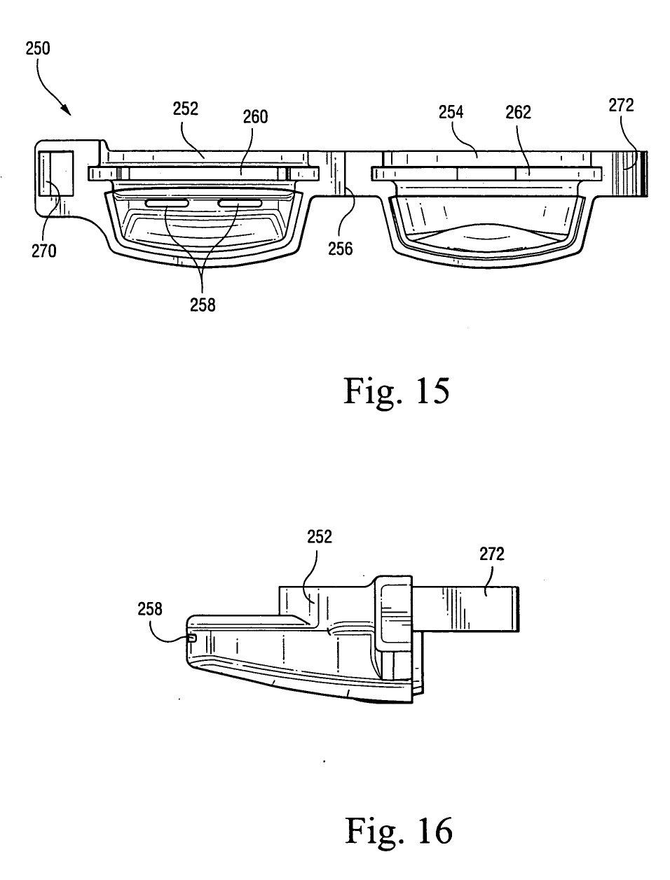

As best shown in FIGS. 11-16, the vent cover 250 includes a first cap portion 252, a second cap portion 254, and an intermediate portion 256 that interconnects the first cap portion 252 and the second cap portion 254. In the illustrated embodiment, the vent cover 250 is molded in one piece and the intermediate portion 256 acts as a living hinge to allow relative movement between the first cap portion 252 and the second cap portion 254. The vent cover 250 may be molded from biocompatible polypropylene, or any other material suitable for snap-fits.

The front face of the first cap portion 252 includes at least one vent aperture 258, e.g., two vent apertures, extending from an interior of the first cap portion 252 to an exterior of the first cap portion 252 for gas washout. In the illustrated embodiment, the vent apertures 258 have a tapered configuration wherein each vent aperture 258 is larger at the entry and smaller at the exit. This configuration helps to minimize noise. However, the vent apertures 258 may have any suitable configuration such as those noted above.

The interior of the first cap portion 252 includes a flange or aperture 260 for engaging the connection flange 24 of the elbow 12. Also, the exterior of the first cap portion 252 includes a first connector in the form of a slot 270 that is adapted to removably engage a second connector provided on the second cap portion 254.

Similar to the first cap portion 252, the second cap portion 254 includes a flange or aperture 262 for engaging the connection flange 24 of the elbow 12. Also, the second connector provided on the second cap portion 254 is in the form of a retaining tab 272. The retaining tab 272 includes a series of ridges 274 to facilitate assembly and/or disassembly with the flange 24 and slot 270.

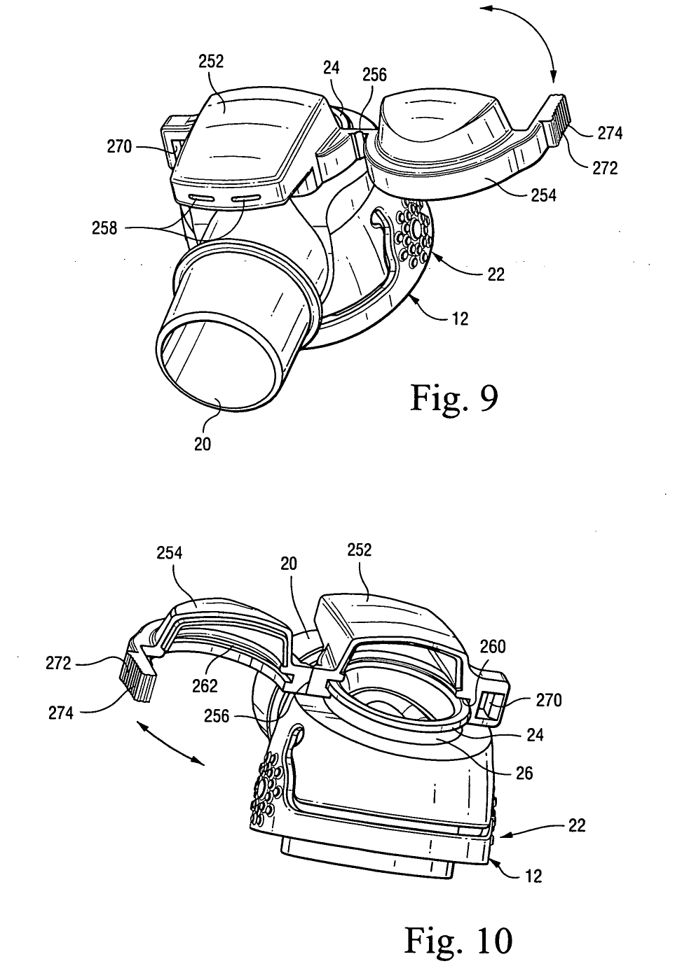

The vent cover 250 may be connected to the elbow 12 in a couple of ways. One way is to initially connect the first cap portion 252 to the second cap portion 254, and then snap the connected cap portions 252, 254 onto the elbow 12. Specifically, the first and second cap portions 252, 254 may be moved toward one another until the retaining tab 272 detachably engages within the slot 270 with a snap-fit. This interlocks the first and second cap portions 252, 254. Then, the connected cap portions 252, 254 are moved into engagement with the flange 24 on the elbow 12 until the flange 24 engages the flanges or apertures 260, 262 provided on the cap portions 252, 254 with a snap-fit. The cap portions 252, 254 may resiliently deform to snap onto and firmly grip the flange 24 when the cap portions 252, 254 reach the operative position as shown in FIGS. 6-8. Also, the cap portions 252, 254 may create an audible clip or snap when properly attached. A seal is maintained by the contact between the edge of the cap portions 252, 254 and the lip of the elbow 12. When desired, the cap portions 252, 254 may be moved into an inoperative position wherein the second cap portion 254 is moved relative to the first cap portion 252 and detached from the elbow 12 as shown in FIGS. 9-10. This allows easy access to the interior vent profile, e.g., for cleaning.

Another way to connect the vent cover 250 to the elbow 12 is to initially connect the first cap portion 252 to the flange 24, and then connect the second cap portion 254 to the first cap portion 252 while on the flange 24. Specifically, the first cap portion 252 is moved into engagement with the flange 24 on the elbow 12 until the flange 24 engages the flange or aperture 260 provided on the first cap portion 252 (e.g., see FIGS. 9 and 10). The first cap portion 252 may be moved into an operative position by sliding the first cap portion 252 into engagement with the flange 24 or by engaging the first cap portion 252 with the flange 24 with a snap-fit. Thus, the first cap portion 24 is supported or captured on the flange 24 of the elbow 12. Then, second cap portion 254 is moved toward the first cap portion 252 until the retaining tab 272 detachably engages within the slot 274 with a snap-fit as shown in FIGS. 6-8. The cap portions 252, 254 may create an audible clip or snap when properly attached. This movement also moves the flange or aperture 262 provided on the second cap portion 254 with the flange 24. When desired, the cap portions 252, 254 may be moved into an inoperative position wherein the second cap portion 254 is moved relative to the first cap portion 252 and detached from the elbow 12, e.g., for cleaning, as shown in FIGS. 9-10.

Also, when attached, the vent cover 250 may be rotated with respect to the flange 24 to adjust the position or direction of the vent apertures 258.

### Alternative Embodiment

In another embodiment the vent may be formed from an additional snap-in piece that is fitted to the vent cover. This has the advantage of allowing more complex vents such as foam, tapered, or sintered vents to be fitted without affecting the manufacture of the vent cover. In particular, this allows the vent cover tool to be designed in such a way that all geometrical features are in a single line of draw, thus removing the need for sliding cores or more complex tooling.

Advantages of Snap-Fit Vent Cover

The snap-fit vent cover configuration allows assembly to be intuitive and quick. That is, it is clear audibly, visually and kinesthetically when the vent cap has “snapped” on. This avoids the problems of softer materials (such as silicone) that require considerable checking to ensure all parts are fitted to the elbow.

This configuration also allows for the vent cover 50, 250 to remain connected to the elbow 12 as a cap portion 52, 254 is detached during cleaning or by inadvertence. That is, the vent cover 50, 250 will not drop and become lost as it is retained by the retaining portion 54 or first cap portion 252.

As noted above, the vent cover 50, 250 may be molded in one piece from polypropylene. This arrangement provides a low cost, single molding alternative to silicone vents that are known in the art. Specifically, significant material cost is saved in using polypropylene instead of liquid silicone rubber (LSR). Also, a polypropylene vent cover has relatively fast production times. Further, a clean blank off is provided at the vent exits of a polypropylene vent cover. This avoids the problem of any flash that is left with silicone molding.

Also, the vent cover may be structured such that the vent cover tool may be designed in such a way that all geometrical features are in a single line of draw, thus removing the need for sliding cores or more complex tooling.

### Other Embodiments

Although the above-described embodiments relate to a vent cover that is attached to an elbow of the mask assembly, it should be understood that the vent cover may be provided along any portion of the gas delivery path. The gas delivery path is any portion of the mask assembly that is adapted to deliver breathable gas to the patient. Thus, the vent cover may be provided on any portion of the mask frame, elbow, and/or even the air delivery tube that delivers breathable gas to the patient. For example, the mask frame, elbow, and/or air delivery tube may include a vent outlet and a connection flange surrounding the vent outlet, and the vent cover may be attached to the connection flange in a manner as described above.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. In addition, while the invention has particular application to patients who suffer from OSA, it is to be appreciated that patients who suffer from other illnesses (e.g., congestive heart failure, diabetes, morbid obesity, stroke, barriatric surgery, etc.) can derive benefit from the above teachings. Moreover, the above teachings have applicability with patients and non-patients alike in non-medical applications.

What is claimed is:

1. A respiratory mask assembly for delivering breathable gas to a patient, comprising:
a gas delivery path defining at least one vent outlet and a connection flange surrounding the vent outlet; and
a vent cover removably connected to the connection flange such that the vent cover is positioned in covering relation with respect to the vent outlet, the vent cover including a cap portion providing at least one vent aperture for gas washout and a ring-shaped retaining portion defining an aperture into which the connection flange is inserted, wherein the cap portion is movably connected to the retaining portion to allow the cap portion to be attached to and detached from the connection flange while the retaining portion remains connected to the connection flange.

2. A respiratory mask assembly according to claim 1, wherein the vent outlet is provided on a mask elbow.

3. A respiratory mask assembly according to claim 2, wherein the elbow is a swivel elbow.

4. A respiratory mask assembly according to claim 1, wherein the vent cover is molded in one-piece.

5. A respiratory mask assembly according to claim 1, wherein the vent cover is molded from polypropylene.

6. A respiratory mask assembly according to claim 1, wherein the cap portion is attached to the connection flange with a snap-fit.

7. A respiratory mask assembly according to claim 6, wherein an interior of the cap portion includes an aperture that is detachably connected to the connection flange.

8. A respiratory mask assembly according to claim 1, wherein the mask assembly is a nasal mask.

9. A respiratory mask assembly according to claim 1, wherein the cap portion includes two vent apertures.

10. A respiratory mask assembly according to claim 1, wherein the cap portion is movably connected to the retaining portion by a living hinge.

11. A respiratory mask assembly for delivering breathable gas to a patient, comprising:
a gas delivery path defining at least one vent outlet and a connection flange surrounding the vent outlet; and
a vent cover removably connected to the connection flange such that the vent cover is positioned in covering relation with respect to the vent outlet,
the vent cover including a first cap portion movably connected to a second cap portion, at least one of the first and second cap portions providing at least one vent aperture for gas washout,
wherein one of the first and second cap portions may be attached to and detached from the connection flange while the other of the first and second cap portions remains connected to the connection flange.

12. A respiratory mask assembly according to claim 11, wherein the vent outlet is provided on a mask elbow.

13. A respiratory mask assembly according to claim 12, wherein the elbow is a swivel elbow.

14. A respiratory mask assembly according to claim 11, wherein the vent cover is molded in one-piece.

15. A respiratory mask assembly according to claim 11, wherein the vent cover is molded from polypropylene.

16. A respiratory mask assembly according to claim 11, wherein one of the first and second cap portions provides a retaining tab that is detachably engagable with a slot provided on the other of the first and second cap portions to interlock the first and second cap portions.

17. A respiratory mask assembly according to claim 16, wherein the first and second cap portions are interlocked with one another and then attached to the connection flange with a snap-fit.

18. A respiratory mask assembly according to claim 17, wherein an interior of each of the first and second cap portions includes an aperture that is detachably connected to the connection flange.

19. A respiratory mask assembly according to claim 16, wherein one of the first and second cap portions is attached to the connection flange prior to the other of the first and second cap portions.

20. A respiratory mask assembly according to claim 11, wherein the mask assembly is a nasal mask.

21. A respiratory mask assembly according to claim 11, wherein the cap portion includes two vent apertures.

22. A respiratory mask assembly according to claim 11, wherein the at least one vent apertures has a tapered configuration.

23. A respiratory mask assembly according to claim 11, wherein the first cap portion is movably connected to the second cap portion by a living hinge.

24. A vent cover for a respiratory mask assembly that delivers breathable gas to a patient, the vent cover comprising:
a cap portion to cover a vent outlet provided along a gas delivery path of the mask assembly, the cap portion including at least one vent aperture for gas washout; and
a ring-shaped retaining portion defining an aperture structured to receive a connection flange surrounding the vent outlet,
wherein the cap portion is movably connected to the retaining portion to allow the cap portion to be attached to and detached from the connection flange while the retaining portion remains connected to the connection flange.

25. A vent cover for a respiratory mask assembly that delivers breathable gas to a patient, the vent cover comprising:
a first cap portion and a second cap portion to cover a vent outlet provided along a gas delivery path of the mask assembly, one of the first and second cap portions providing at least one vent aperture for gas washout,
wherein the first cap portion is movably mounted to the second cap portion to allow one of the first and second cap portions to be attached to and detached from the connection flange while the other of the first and second cap portions remains connected to the connection flange.

26. A respiratory mask assembly for delivering breathable gas to a patient, comprising:
a gas delivery path defining at least one vent outlet and a connection flange surrounding the vent outlet; and
a vent cover molded in one piece from polypropylene and having a first portion movably connected to a second portion by a living hinge, one of the first and second portions providing at least one vent aperture for gas washout.

27. A respiratory mask assembly according to claim 26, wherein the vent outlet is provided on a mask elbow.

28. A respiratory mask assembly for delivering breathable gas to a patient, comprising:
a gas delivery path defining at least one vent outlet and a connection flange surrounding the vent outlet; and
a vent cover having at least one vent aperture for gas washout and being detachably connectable to the connection flange with a snap-fit.

29. A respiratory mask assembly according to claim 28, wherein the vent outlet is provided on a mask elbow.

30. A method for mounting a vent cover to a respiratory mask assembly that delivers breathable gas to a patient, the method comprising:
providing first and second vent portions;
positioning the first vent portion adjacent to a connection flange surrounding a vent outlet of the mask assembly; and
selectively connecting the second vent portion to the connection flange by moving the second vent portion relative to the first vent portion.

31. The method according to claim 30, wherein the second vent portion is selectively connected to the connection flange while selectively connecting the second vent portion to the first vent portion.

32. The method according to claim 30, wherein the second vent portion is selectively connected to the first vent portion prior to selectively connecting the first and second vent portions to the connection flange.

33. The method according to claim 30, wherein the first vent portion is a ring-shaped retaining portion that defines an aperture into which the connection flange is inserted.
